(19) United States
(12) Patent Application Publication
(10) Pub. No.: US 2018/0230421 A1
(43) Pub. Date: Aug. 16, 2018

(54) METHOD FOR THE PROTEIN ENRICHMENT OF MICROALGAL BIOMASS

(71) Applicant: Corbion Biotech, Inc., South San Francisco, CA (US)

(72) Inventors: Gabriel Macquart, Mont Bernanchon (FR); Sylvain Delaroche, Longuenesse (FR); Marie Le Ruyet, Lille (FR); Laurent Segueilha, Marquette Lez Lille (FR)

(21) Appl. No.: 15/953,312

(22) Filed: Apr. 13, 2018

Related U.S. Application Data
(63) Continuation of application No. 15/112,436, filed on Jul. 19, 2016, now abandoned.

(30) Foreign Application Priority Data
Jan. 20, 2014 (FR) 1450419

(51) Int. Cl. C12N 1/38; C12P 13/14; C12P 21/00; C12P 13/10; C12N 1/12

(52) U.S. Cl. CPC C12N 1/38; C12P 13/14; C12P 21/00; C12N 1/12; C12P 13/10

(57) ABSTRACT

The invention relates to a method for the protein enrichment of a microalga of the genus Chlorella, cultivated under heterotrophic conditions. The method is characterized in that the heterotrophic cultivation comprises a step intended to slow down the growth of the microalga, with the fermentation medium being deficient in a nitrogen-free nutritional source.

The present invention relates to a process for the protein enrichment of microalgal biomass, more particularly of the Chlorella genus, even more particularly of the species Chlorella sorokiniana or Chlorella protothecoides. The present invention also relates to a process for the protein enrichment of the biomass of certain microalgae, more particularly of Chlorella protothecoides, the arginine and glutamine content of which proteins is remarkably high.

Macroalgae and microalgae have a specific richness which remains largely unexplored. Their utilization for dietary, chemical or bioenergy purposes is still highly marginal. However, they contain components of great value, in terms of both richness and abundance.

Indeed, microalgae are sources of vitamins, lipids, proteins, sugars, pigments and antioxidants.

Algae and microalgae are thus of interest to the industrial sector, where they are used for manufacturing food supplements, functional foods, cosmetics and medicaments, or for aquaculture.

Microalgae are first and foremost photosynthetic microorganisms which colonize all biotopes exposed to light.

On the industrial scale, the monoclonal culturing thereof is performed in photobioreactors (autotrophic conditions: in light with CO₂) or, for some, it is also performed in fermenters (heterotrophic conditions: in darkness in the presence of a source of carbon).

This is because some species of microalgae are able to grow in the absence of light: Chlorella, Nitzschia, Cyclotella, Tetraselmis, Crypthecodinium, Schizochytrium.

Moreover, it is estimated that culturing under heterotrophic conditions is 10 times less expensive than under phototrophic conditions because, for those skilled in the art, these heterotrophic conditions allow:
the use of fermenters identical to those used for bacteria and yeast, enabling all the culturing parameters to be controlled,
the production of biomasses in much greater amounts than those obtained by light-based culturing.

The profitable utilization of microalgae generally necessitates controlling the fermentation conditions, making it possible to accumulate their components of interest, such as:
pigments (chlorophyll a, b and c, β-carotene, astaxanthin, lutein, phycocyanin, xanthophylls, phycoerythrin, etc.), the demand for which is increasing both due to their noteworthy antioxidant properties and to their provision of natural colorings for food,
lipids, in order to optimize their content of fatty acids (up to 60%, or even 80% by weight of their solids), especially for:
   biofuel applications, but also
   applications in food for human consumption or animal feed, when the selected microalgae produce “essential” (i.e. supplied by the diet because they are not naturally produced by humans or animals) polyunsaturated fatty acids or PUFAs.
proteins, in order to optimize the nutritive qualities thereof or, for example, to promote the supply of amino acids of interest.

In the context of supplying amino acids of interest, it may in fact be advantageous to have available protein sources that are rich in arginine and glutamate.

Arginine is an amino acid that has many functions in the animal kingdom.

Arginine may be degraded and may thus serve as a source of energy, carbon and nitrogen for the cell which assimilates it.

In various animals, including mammals, arginine is decomposed into ornithine and urea. The latter is a nitrogenous molecule that can be eliminated (via excretion in the urine) so as to regulate the amount of nitrogenous compounds present in the cells of animal organisms.

Arginine allows the synthesis of nitrogen monoxide (NO) via NO synthetase, thus participating in the vasodilation of the arteries, which reduces the rigidity of the blood vessels, increases the blood flow and thus improves the functioning of the blood vessels.

Food supplements which contain arginine are recommended for promoting the health of the heart, the vascular function, for preventing “platelet aggregation” (risk of formation of blood clots) and for lowering the arterial pressure.

The involvement of arginine in the healing of wounds is associated with its role in the formation of proline, which is another important amino acid in collagen synthesis.

Finally, arginine is a component that is frequently used, especially by sportspeople, in energy drinks.

As regards glutamic acid, it is not only one of the elementary bricks used for protein synthesis, but is also the excitatory neurotransmitter that is the most widespread in the central nervous system (encephalon+spinal column) and is a GABA precursor in GABAergic neurons.

Under the code E620, glutamate is used as a flavor enhancer in foods. It is added to food preparations to enhance their taste.

Besides glutamate, the Codex Alimentarius has also recognized as flavor enhancers the sodium salt (E621), the potassium salt (E622), the calcium salt (E623), the ammonium salt (E624) and the magnesium salt (E625) thereof.

Glutamate (or the salts thereof) is often present in ready-made meals (soups, sauces, crisps and ready-made dishes). It is also commonly used in Asian cookery.

It is currently frequently used in combination with flavorings in aperitifs (bacon flavor, cheese flavor). This makes it possible to enhance the bacon, cheese, etc. flavor. It is rare to find an aperitif not containing any.

It is also found in certain medicament capsules, but not for its taste functions.

Finally, it is the major component of cooking auxiliaries (stock cubes, sauce bases, sauces, etc.).

In order to utilize the metabolic richness of microalgae, first fermentation methods for obtaining high cell densities (HCD) were thus thoroughly investigated so as to obtain maximum protein or lipid yields and productivity.

The aim of these HCD cultures was to obtain the highest possible concentration of the desired product in the shortest possible period of time.

This principle is borne out for example by the biosynthesis of astaxanthin by Chlorella zofingiensis, in which growth of the microalga proved to be directly correlated with the production of this compound (Wang and Peng, 2008, World J. Microbiol. Biotechnol., 24 (9) , 1915-1922) .

However, maintaining growth at its maximum rate (μ, in h⁻¹) is not always correlated with high production of the desired product.

Indeed, it quickly became apparent to specialists in the field that it is necessary, for example, to subject the microalgae to a nutritional stress which limits their growth, when it is desired to make them produce large lipid reserves.

Thus, in fermenting methods, growth and production were uncoupled.

For example, to promote the accumulation of polyunsaturated fatty acids (in this instance docosahexaenoic acid or DHA), patent application WO 01/54510 recommends dissociating cell growth from the production of polyunsaturated fatty acids.

More particularly, a method for producing microbial lipids is claimed therein, which method comprises the steps consisting in:

(a) performing fermentation of a medium comprising microorganisms, a carbon source and a limiting nutritional source, and ensuring conditions sufficient to maintain a dissolved oxygen content of at least approximately 4% of saturation in said fermentation medium to increase the biomass;

(b) then providing conditions sufficient to maintain a dissolved oxygen content of approximately less than or equal to 1% of saturation in said fermentation medium and providing conditions sufficient to allow said microorganisms to produce said lipids;

(c) and collecting said microbial lipids, in which at least approximately 15% of said microbial lipids are constituted of polyunsaturated lipids;

and in which a biomass density of at least approximately 100 g/l is obtained over the course of the fermentation.

In the microalga Schizochytrium sp., strain ATCC 20888, a first growth phase is thus more particularly performed in the presence of a carbon source and a nitrogen source but without limiting oxygen, so as to promote the production of a high cell density, then, in a second phase, the supply of nitrogen is stopped and the supply of oxygen is gradually slowed (management of the dissolved oxygen pressure or pO₂ from 10% to 4% then to 0.5%), so as to stress the microalga, slow its growth and trigger production of the fatty acids of interest.

In the microalga Crypthecodinium cohnii, the highest content of docosahexaenoic acid (DHA, a polyunsaturated fatty acid) is obtained at low glucose concentration (of the order of 5 g/l) and thus at a low growth rate (Jiang and Chen, 2000, Process Biochem., 35(10), 1205-1209).

These results are a good illustration of the fact that the product formation kinetics can be associated both positively and negatively with growth of the microalgae, or even a combination of the two.

Consequently, in the event that the formation of products is not correlated with high cell growth, it is prudent to control the rate of cell growth.

In general, those skilled in the art choose to control the growth of the microalgae by controlling the fermentation conditions (temperature, pH) or by regulated feeding of nutritional components to the fermentation medium (semicontinuous conditions referred to as “fed batch”).

If they choose to control the growth of the microalgae heterotrophically through the supply of carbon sources, those skilled in the art generally choose to adapt the carbon source (pure glucose, acetate, ethanol, etc.) to the microalga (C. cohnii, Euglena gracilis, etc.) as a function of the metabolite produced (for example a polyunsaturated fatty acid of DHA type).

Temperature may also be a key parameter:
for example, it has been reported that the synthesis of polyunsaturated fatty acids in some species of microalgae, such as EPA by Chlorella minutissima, is promoted at a lower temperature than that required for the optimal growth of said microalga;
on the other hand, the lutein yield is higher in heterotrophically cultivated Chlorella protothecoides when the production temperature is increased from 24 to 35° C.

Indeed, Chlorella protothecoides is acknowledged to be one of the best oil-producing microalgae.

Under heterotrophic conditions, it rapidly converts carbohydrates to triglycerides (more than 50% of the solids thereof).

To optimize this production of triglycerides, those skilled in the art are led to optimize the carbon flow toward oil production, by acting on the nutritional environment of the fermentation medium.

Thus, it is known that oil accumulates when there is a sufficient supply of carbon but under conditions of nitrogen deficiency.

Therefore, the C/N ratio is the determining factor here, and it is accepted that the best results are obtained by acting directly on the nitrogen content, with the glucose content not being a limiting factor.

Unsurprisingly, this nitrogen deficiency affects cell growth, which results in a growth rate 30% lower than the normal growth rate for the microalga (Xiong et al., Plant Physiology, 2010, 154, pages 1001-1011).

To explain this result, in the abovementioned article Xiong et al. in fact demonstrate that if the Chlorella biomass is divided into its 5 main components, i.e. carbohydrates, lipids, proteins, DNA and RNA (representing 85% of the solids thereof), while the C/N ratio has no impact on the content of DNA, RNA or carbohydrates, it becomes paramount for the content of proteins and lipids.

Thus, Chlorella cells cultivated with a low C/N ratio contain 25.8% proteins and 25.23% lipids, whereas a high C/N ratio makes the synthesis of 53.8% lipids and 10.5% proteins possible.

To optimize its oil production, it is therefore essential for those skilled in the art to control the carbon flow by steering it toward oil production to the detriment of protein production; the carbon flow is redistributed and accumulates as lipid storage substances when the microalgae are placed in a nitrogen-deficient medium.

Armed with this teaching, in order to produce protein-rich biomasses, those skilled in the art are therefore led to perform the opposite of this metabolic control, i.e. to modify the fermentation conditions by instead promoting a low C/N ratio, and thus:
supply a large amount of nitrogen source to the fermentation medium while keeping constant the carbon source feedstock, which will be converted into proteins, and
stimulate the growth of the microalga.

This involves modifying the carbon flow toward protein (and hence biomass) production, to the detriment of storage lipid production.

Within the context of the invention, the Applicant Company has, on the other hand, chosen to explore an original route by proposing an alternative solution to that conventionally envisioned by those skilled in the art.

The invention thus relates to a process for the protein enrichment of a microalga cultured heterotrophically, the microalga being of the genus Chlorella, even more particularly Chlorella sorokiniana or Chlorella protothecoides, the heterotrophic culture process comprising a step directed toward limiting the growth of said microalga via deficiency of the fermentation medium in a non-nitrogen nutritional source.

This step is a heterotrophic culture step in which a non-nitrogen nutritive factor is supplied in insufficient amount to the medium to allow growth of the microalga.

For the purposes of the invention, the term “enrichment” means an increase in the protein content of the biomass of at least 15%, preferably at least 20% by weight, so as to reach a protein content of the biomass of more than 50%, 60%, 65% or 70% by weight.

The invention more precisely covers a process for the heterotrophic culturing of said microalgae, comprising a step directed toward limiting the growth of said microalga via a deficiency of the fermentation medium in a non-nitrogen nutritional source. Optionally, the fermentation temperature may also be modified so as to slow down the growth of the microalga. The fermentation temperature may especially be increased by 1, 2, 3, 4 or 5 degrees relative to the optimum fermentation temperature, which is usually about 28° C. Preferably, the fermentation temperature is increased by about 3° C., for example going from 28° C. to 31° C.

As it is used here, the term “about” refers to a value ±20%, 10%, 5% or 2%. The present invention thus relates to a process for the protein enrichment of a microalga cultured heterotrophically, the microalga being of the genus Chlorella, even more particularly Chlorella sorokiniana or Chlorella protothecoides, the process comprising heterotrophically culturing, which comprises a step directed toward limiting the growth of said microalga via deficiency of the fermentation medium in a non-nitrogen nutritional source, thus making it possible to reach a protein content of the biomass of more than 50%, 60% or 70% by weight.

The term “deficiency of the fermentation medium in a non-nitrogen nutritional source” means culturing in which at least one of the non-nitrogen nutritive factors is supplied to the microalga in an insufficient amount to allow its growth. This deficiency is reflected by a growth rate of the microalga that is below that of said microalga in the absence of nutritional limitation.

This is reflected by an absence of non-nitrogen residual nutritive factor in the culture medium, the microalga consuming this nutritive factor gradually as it is supplied.

For the purposes of the invention, the essential criterion is thus limitation of the cell growth induced by a stress, the cellular stress brought about by the deficiency of a non-nitrogen nutritive substance of the fermentation medium.

This strategy therefore goes heavily against the technical preconception which considers that to increase the content of proteins in the biomass, it is absolutely imperative to increase this biomass and therefore the cell growth.

The term “a non-nitrogen nutritional source” means a nutritive substance chosen, for example, from the group constituted by glucose and phosphates.

As will be illustrated below, it may advantageously be chosen to limit the growth of:
Chlorella sorokiniana via a glucose deficiency of the fermentation medium,
Chlorella protothecoides via a phosphate deficiency of the fermentation medium.

Optionally, the growth limitation of said microalga may be obtained by adding to the culture medium substances that inhibit cell growth, such as sulfates.

Slowing down the growth of said microalga may also be elicited by modifying the fermentation temperature, for example by increasing it by about 1 to 5° C., preferably by about 3° C., relative to the optimum fermentation temperature. This optimum fermentation temperature is usually about 28° C.

Moreover, without being bound by any theory, the Applicant Company has found that the glucose flow is normally used in microalgae of the genus Chlorella sorokiniana in a quite specific order of priority:
1. basal metabolism,
2. growth, i.e. formation of a protein-rich biomass,
3. storage substances (fats and carbohydrates such as starch).

This principle explains the natural variations in the protein content in the course of growth of the microalga, despite the constant supply of nitrogen.

The Applicant Company has thus found that, in order to enrich the microalgal biomass with proteins, the growth of the microalga needs to be limited and its consumption of nutritive source other than nitrogen, for example of glucose, needs to be controlled so as to:
dedicate the glucose consumption entirely to the protein production pathways,
avoid the accumulation of storage substances such as fats.

Specifically, avoiding a nitrogen deficiency makes it possible to prevent the metabolic flows from being diverted toward fat production.

Optionally, it may be advantageous to go as far as completely blocking any synthesis of storage material, by acting via specific inhibitors.

Specifically, a certain number of inhibitors of fat or even starch (primordial storage carbohydrate of green microalgae) synthesis pathways are known:
for fats, cerulenin is described as an inhibitor of fatty acid synthesis, or lipstatin, a natural substance produced by Streptomyces toxytricini, is described as a lipase inhibitor, etc.
for starch, imino sugars (obtained by simple substitution of the endocyclic oxygen atom of sugars with a nitrogen atom) are historically known as potent inhibitors of glycosidases, glycosyltransferases, glycogen phosphorylases, or UDP-Galp mutase.

Thus, the process comprises the fermentation of a microalgal biomass under heterotrophic conditions with a first step of growth of the biomass and with a second step of deficiency of the fermentation medium in a non-nitrogen nutritional source.

This second step makes it possible to enrich the biomass with protein. In particular, it makes it possible to achieve a protein content of the biomass of more than 50%, 60%, 65% or 70% by weight (by weight of solids).

In a first preferred embodiment in accordance with the invention, the process for heterotrophic culturing of said microalgae, especially Chlorella sorokiniana, comprises:
a first step of growth of the microalgae, in which the amount of glucose in the medium (in batch mode) or the glucose supply rate (in continuous or semi-continuous mode) is adjusted to their consumption capacity, so as rapidly to obtain a large biomass,
a second step of protein production by the microalgae, in which the glucose supply rate is set at a value markedly below their glucose consumption capacity, so as to prevent the additional accumulation of storage substances, or even to promote their consumption.

As will be illustrated below, the first step of growth of the microalgae may be performed in batch mode, in which the glucose initially supplied is entirely consumed by the microalga, leading to the production of a base biomass.

The second step of protein production is performed under conditions in which:

a) either whole medium is supplied in semi-continuous or “fed-batch” mode, after consumption of the glucose initially supplied; the other parameters for performing the fermentation are unchanged. In this case, glucose is supplied continuously and the supply rate is then less than the consumption rate that the strain might achieve, such that the residual glucose content in the medium is maintained at zero. The growth of the strain is then limited by the availability of glucose (glucose-limiting condition).

b) continuous functioning of chemostat type, in which the growth rate of the strain (μ) is maintained at its minimum value, the growth of the strain being limited by the glucose supply. This mode of functioning makes it possible to obtain biomass with a high protein content by means of glucose limitation and the low growth rate imposed, while at the same time ensuring very good productivity.

In a second preferred embodiment in accordance with the invention, the process for heterotrophic culturing of said microalgae, especially Chlorella protothecoides, comprises a step of growth of the microalgae in which limitation of the phosphate supply limits the growth rate and results in an increase in the protein content. Thus, heterotrophic culturing of microalgae of the species Chlorella protothecoides comprises a step of heterotrophic culturing with a phosphate deficiency, the growth rate thus being reduced and resulting in an increase in the protein content.

In one variant of this second preferred embodiment in accordance with the invention, the fermentation temperature during this growth step is increased by about 1 to 5° C., preferably by about 3° C., relative to the optimum fermentation temperature. The optimum temperature is usually about 28° C. According to a particularly preferred embodiment, the fermentation temperature during the growth step is about 31° C.

Thus, according to a particular embodiment, the heterotrophic culturing of the microalgae of the species Chlorella protothecoides comprises a step of heterotrophic culturing at a higher temperature than the optimum temperature, which is about 28° C., with a phosphate deficiency. This embodiment makes it possible to further reduce the growth rate and thus to further increase the protein content.

As will be illustrated below, increasing the fermentation temperature makes it possible to increase the cooling capacity of the heat exchanger of the fermenter. An increase in fermentation temperature from 28° C. to 31° C. thus makes it possible to double the cooling capacity of the heat exchanger of the fermenter.

Moreover, these conditions directed toward limiting the growth of the microalgae of the species Chlorella protothecoides, by limiting the availability of nutrients other than nitrogen, are reflected here not only by an increase in the protein richness, but also lead to appreciably increasing the arginine and glutamate content thereof.

Without being bound by any theory, the Applicant Company considers that limitation of the growth of these particular microalgae is reflected by a slowing-down of their overall metabolic activity (which has an impact on the growth rate) and the accumulation of molecules rich in C and N (of amino acid type), the biosynthetic pathways of which best “withstand” the nutritional deficiency.

In Chlorella protothecoides, the molecules concerned will be arginine and glutamate.

More particularly, as will be illustrated below, the heterotrophic culturing of microalgae of the species Chlorella protothecoides comprising a step of culturing with a phosphate deficiency, which limits the growth rate and results in an increase in the protein content, thus leads to producing more than 40% of glutamic acid and of arginine out of the total amino acids.

Thus, the present invention also relates to a process for enriching the glutamic acid and/or arginine content of a heterotrophically cultivated microalga, preferably of a microalga of the species Chlorella protothecoides, the process comprising heterotrophic culturing of the microalgae, preferably of the species Chlorella protothecoides, comprising a step of culturing with a phosphate deficiency, this culturing resulting in the preparation of a microalgal biomass comprising more than 40% of glutamic acid and of arginine out of the total amino acids. Preferably, this process also makes it possible to enrich the content of glutamic acid and of arginine. In particular, this process also makes it possible to enrich the protein content. Thus, the biomass produced preferably comprises more than 60%, 65% or 70% by weight of protein dry matter. In one embodiment, the fermentation temperature is about 28° C. In another embodiment, the fermentation temperature is increased by 1, 2, 3, 4 or 5° C. relative to the optimum fermentation temperature. It is preferably about 31° C.

The present invention relates to biomass produced via this enrichment process. The invention also relates to biomass that may be obtained via this process. Said biomass comprises more than 60%, 65% or 70% by dry weight of protein and more than 40% of glutamic acid and of arginine out of the total amino acids. In particular, the biomass comprises 40%, 45%, 50%, 55% 60% or 65% or more of glutamic acid and of arginine out of the total amino acids. More particularly, the biomass may comprise 20%, 25%, 30% or 35% or more of glutamic acid out of the total amino acids. It may comprise 20%, 25% or 30% or more of arginine out of the total amino acids. The biomass is a biomass of Chlorella protothecoides.

In one particular embodiment, it is the proteins whose richness in glutamic acid and/or in arginine, preferably in glutamic acid and in arginine, which are increased.

The invention will be understood more clearly from the following examples which are intended to be illustrative and nonlimiting.

### EXAMPLES

### Example 1: Production of Chlorella sorokiniana in Fermentation of “Sequential Batch” Type without Limitation of the Supply of Nutrient Medium

The strain used is a Chlorella sorokiniana (strain UTEX 1663—The Culture Collection of Algae at the University of Texas at Austin-USA).

Preculture:
600 ml of medium in a 2 L conical flask;
Composition of the medium (table 1 below).

TABLE 1

| | | |
|---|---|---|
| Macro | Glucose | 20 |
| elements | K₂HPO₄•3H₂O | 0.7 |
| (g/l) | MgSO₄•7H₂O | 0.34 |
| | Citric acid | 1.0 |
| | Urea | 1.08 |
| | Na₂SO₄ | 0.2 |
| | Na₂CO₃ | 0.1 |
| | clerol FBA 3107 (antifoam) | 0.5 |
| Micro | Na₂EDTA | 10 |
| elements | CaCl₂2H₂O | 80 |
| (mg/l) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

The pH is adjusted to 7 before sterilization by addition of 8N NaOH.

Incubation is performed under the following conditions:
duration: 72 h;
temperature: 28° C.;
shaking: 110 rpm (Infors Multitron incubator).

The preculture is then transferred to a 30 L Sartorius type fermenter.

Culture for Biomass Production:

The medium is identical to that of the preculture, but the urea is replaced with NH₄Cl.

TABLE 2

| | | |
|---|---|---|
| Macro | Glucose | 20 |
| elements | K₂HPO₄•3H₂O | 0.7 |
| (g/l) | MgSO₄•7H₂O | 0.34 |
| | Citric acid | 1.0 |
| | NH₄Cl | 1.88 |
| | Na₂SO₄ | 0.2 |
| | clerol FBA 3107 (antifoam) | 0.5 |
| Micro | Na₂EDTA | 10 |
| elements | CaCl₂ | 80 |
| (mg/l) | FeSO₄•7H₂O | 40 |
| | MnSO₄•4H₂O | 0.41 |
| | CoSO₄•7H₂O | 0.24 |
| | CuSO₄•5H₂O | 0.24 |
| | ZnSO₄•7H₂O | 0.5 |
| | H₃BO₃ | 0.11 |
| | (NH₄)₆Mo₇O₂₇•4H₂O | 0.04 |

The initial volume (Vi) of the fermenter is adjusted to 13.5 L after inoculation.

It is finally brought to 16-20 L.

The parameters for performing the fermentation are as follows:

TABLE 3

| | |
|---|---|
| Temperature | 28° C. |
| pH | 5.0-5.2 by 28% w/w NH₃ |
| pO₂ | >20% (maintained by shaking) |
| Shaking | Minimum 300 rpm |
| Air flow rate | 15 l/min |

When the glucose initially supplied is consumed, medium identical to the initial medium, without the antifoam, is supplied in the form of a concentrated solution containing 500 g/L of glucose and the other elements in the same proportions relative to the glucose as in the initial medium, so as to obtain a glucose content of 20 g/L in the fermenter.

Two other identical additions are performed in the same manner each time that the residual glucose concentration becomes zero.

Clerol FBA 3107 antifoam is added as required to avoid excessive foaming.

Results:

After 46 hours of culturing, 38 g/L of biomass with a protein content (evaluated by the N 6.25) of 36.2% are obtained.

### Example 2: Production of C. sorokiniana in Fermentation of Fed-Batch Type with Limiting Glucose Supply

In this example, a supply of whole medium (fed-batch mode) is started after consumption of the glucose initially supplied. The other parameters for performing the fermentation are unchanged.

Glucose is supplied continuously using a 500 g/L concentrated solution. The supply rate is less than the consumption rate that the strain might achieve, such that the residual glucose content in the medium is kept at zero, i.e. the growth of the strain is limited by the glucose availability (glucose-limiting condition).

This rate is increased exponentially over time. The formula for calculating the supply rate is characterized by a factor μ which corresponds to the growth rate that the strain can adopt if it consumes all of the glucose supplied:

S=So×exp (μ.t)

S=glucose supply rate (in g/h)

So=initial glucose supply rate, determined as a function of the biomass present at the end of the batch. It is 12 g/h under our conditions.

μ=rate acceleration factor. It should be less than 0.11 h⁻¹, which is the growth rate of the strain in the absence of nutritional limitation.

t=fed-batch time (in h)

The salts are supplied if possible continuously, separately or mixed with the glucose. However, they may also be supplied sequentially in several portions.

Table 4 below gives the salt needs per 100 g of glucose.

TABLE 4

| | | |
|---|---|---|
| Macro | Glucose | 100 |
| elements | K₂HPO₄•3H₂O | 6.75 |
| (g) | MgSO₄•7H₂O | 1.7 |
| | Citric acid | 5.0 |
| | Na₂SO₄ | 1.0 |
| Micro | Na₂EDTA | 50 |
| elements | CaCl₂2H₂O | 400 |
| (mg) | FeSO₄•7H₂O | 200 |
| | MnSO₄•4H₂O | 2.1 |
| | CoSO₄•7H₂O | 1.2 |
| | CuSO₄•5H₂O | 1.2 |
| | ZnSO₄•7H₂O | 2.5 |
| | H₃BO₃ | 0.6 |
| | (NH₄)₆Mo₇O₂₇•4H₂O | 0.2 |

The concentrations of the elements other than the glucose were determined so that they were in excess relative to the nutritional requirements of the strain.

Clerol FBA 3107 antifoam is added as required to avoid excessive foaming.

Results: Effect of the Glucose Supply Rate in the Fed-Batch Mode

Tests were performed at various glucose supply rates in fed-batch mode. They are characterized by the μ applied. The protein content of the biomass obtained is evaluated by measuring the total nitrogen expressed by N 6.25.

TABLE 5

| | μ fed | Duration | Biomass | Productivity | % N |
|---|---|---|---|---|---|
| Test | (h⁻¹) | (h) | (g/l) | (g/L/h) | 6.25 |
| 1 | 0.06 | 78 | 43.6 | 0.56 | 49.2 |
| 2 | 0.07 | 54 | 35.1 | 0.65 | 43.1 |
| 3 | 0.09 | 48 | 64.9 | 1.35 | 39.3 |

These results show that working under glucose-limiting conditions makes it possible to increase the protein content.

Specifically, it is observed that, even with a high μ of 0.09, a protein content higher than that obtained without limitation as in Example 1 (39.3% instead of 36.2%) is obtained.

Increasing the limitation of the metabolism with glucose results in an additional improvement in the protein content.

Under the conditions of these tests, it is necessary to impose on the strain a μ of less than 0.06 h⁻¹ to obtain a protein content of greater than 50%.

It should be noted that this condition goes hand in hand with a reduction of the productivity: 0.56 g/L/h instead of 1.35 g/L/h with Test 3.

### Example 3: Production of C. sorokiniana in Continuous Fermentation of Chemostat Type with Limiting Glucose Supply

In this example, the fermenter used is a 2L Sartorius Biostat B fermenter.

The fermentation is performed as in Example 2, but with ten times smaller volumes: the inoculum is 60 ml and the initial volume is 1.35 L.

Continuous supply of the medium is started according to the same principle as in Example 2, the salts in this case being mixed with the glucose in the feed manifold. The supply rate is accelerated according to the same exponential formula as in Example 2 by applying a p of 0.06 h⁻¹.

Chemostat

When a volume of 1.6 L is reached, i.e. a biomasses concentration of about 50 g/L, continuous functioning of chemostat type is implemented:
1. The fermenter is fed continuously at a rate of 96 ml/h with a nutrient medium solution containing 100 g/L of glucose, having the following composition:

TABLE 6

| | | |
|---|---|---|
| Macro | Glucose | 100 |
| elements | K₂HPO₄•3H₂O | 6.75 |
| (g/l) | MgSO₄•7H₂O | 1.7 |
| | Citric acid | 5.0 |
| | Na₂SO₄ | 1.0 |
| Micro | Na₂EDTA | 50 |
| elements | CaCl₂2H₂O | 400 |
| (mg/l) | FeSO₄•7H₂O | 200 |
| | MnSO₄•4H₂O | 2.1 |
| | CoSO₄•7H₂O | 1.2 |
| | CuSO₄•5H₂O | 1.2 |
| | ZnSO₄•7H₂O | 2.5 |
| | H₃BO₃ | 0.6 |
| | (NH₄)₆Mo₇O₂₇•4H₂O | 0.2 |

The concentrations of the elements other than the glucose were determined so that they were in excess relative to the nutritional requirements of the strain.
2. Medium is withdrawn continuously from the fermenter via a dip tube connected to a pump so as to keep the volume of the culture at 1.6 L.

Thus, the medium is renewed with a 0.06 (6%) fraction per hour. This renewal rate is referred to as the dilution rate (D).

In accordance with the principle of chemostat culturing, the growth rate of the strain (μ) becomes established at the same value since the growth of the strain is limited by the glucose supply:

D=μ=0.06 h⁻¹

Results

After 97 hours of chemostat functioning, the biomasses concentration becomes established at 48 g/L±2 g/L and the protein content at 53±2%.

This mode of functioning makes it possible to obtain biomass with a high protein content by means of glucose limitation and the low growth rate imposed, while at the same time ensuring very good productivity, of the order of 2.9 g/L/h, by means of the high concentration of biomass.

### Example 4: Production of Chlorella protothecoides in Fermentation of Batch Type with or without Limitation of the Phosphate Supply

The strain used is a Chlorella protothecoides (strain CCAP211/8D—The Culture Collection of Algae and Protozoa, Scotland, UK).

Preculture:
150 ml of medium in a 500 L conical flask;
Composition of the medium: 40 g/L of glucose+10 g/l of yeast extract.

Incubation is carried out under the following conditions: duration: 72 h; temperature: 28° C.; stirring: 110 rpm (Infors Multitron incubator).

The preculture is then transferred into a 2 L Sartorius Biostat B fermenter.

Culture for Biomass Production:

The composition of the culture medium is as follows (in g/L):

TABLE 7

| | |
|---|---|
| Glucose | 80 |
| Citric acid | 4 |
| NH₄Cl | 2 |
| KH₂PO₄ | 3 (test 1)/2 (test 2)/1 (test 3) |
| Na₂HPO₄ | 3 (test 1)/2 (test 2)/1 (test 3) |
| MgSO₄•7H₂O | 1.5 |
| NaCl | 0.5 |
| Yeast extract | 5 |

The phosphate supply is calculated so that it is in excess in test 1 and limiting for tests 2 and 3.

Clerol FBA 3107 antifoam is added as required to avoid excessive foaming.

The initial volume (Vi) of the fermenter is adjusted to 1 L after inoculation.

The parameters for performing the fermentation are as follows:

TABLE 8

| | |
|---|---|
| Temperature | 28° C. |
| pH | 6.5 by NH₃ 28 ww % |
| pO₂ | >20% (maintained by shaking) |
| Shaking | Minimum 200 rpm |
| Air flow rate | 1 L/min |

Results:

TABLE 9

| | | | | Residual | |
|---|---|---|---|---|---|
| | Duration | Biomass | Cumulative | PO₄ | % N |
| Test | (h) | (g/l) | μ (h⁻¹) | (mg/L) | 6.25 |
| 1 | 36 | 38.1 | 0.09 | 800 | 48.1 |
| 2 | 45 | 36.5 | 0.07 | 0 | 56.1 |
| 3 | 54 | 36 | 0.05 | 0 | 61.2 |

The cumulative μ value corresponds to the growth rate of the biomass from inoculation.

The protein content is estimated by measuring the nitrogen content N×6.25

These results show that a limitation of the phosphate supply, confirmed by the absence of residual phosphate at the end of fermentation, limits the growth speed (measured by the growth rate) and, as for the glucose limitation in the preceding examples, results in an increase in the protein content to reach values markedly greater than 50%.

### Example 5: Production of Chlorella protothecoides at 28° C. in Fermentation of Fed-Batch Type Using a Phosphate-Limited Synthetic Medium

To obtain a high biomasses concentration, glucose is supplied during culturing (fed-batch) to avoid growth inhibition by glucose.

The supplies of salts, in particular phosphate, are conventionally performed at the start of fermentation (batch mode).

The culture medium is free of yeast extract.

As in Example 4, the strain used is a Chlorella protothecoides (strain CCAP211/8D—The Culture Collection of Algae and Protozoa, Scotland, UK).

Preculture:
500 ml of medium in a 2 L conical flask;
Composition of the medium:

TABLE 10

| | | |
|---|---|---|
| Macro | Glucose | 40 |
| elements (g/l) | K₂HPO₄ | 3 |
| | Na₂HPO₄ | 3 |
| | MgSO₄•7H₂O | 0.25 |
| | (NH₄)₂SO₄ | 1 |
| | Citric acid | 1 |
| | clerol FBA 3107 (antifoam) | 0.1 |
| Microelements | CaCl₂•2H₂O | 30 |
| and Vitamins | FeSO₄•7H₂O | 1 |
| (mg/l) | MnSO₄•1H₂O | 8 |
| | CoSO₄•7H₂O | 0.1 |
| | CuSO₄•5H₂O | 0.2 |
| | ZnSO₄•7H₂O | 0.5 |
| | H₃BO₃ | 0.1 |
| | Na₂MoO₄•2H₂O | 0.4 |
| | Thiamine HCl | 1 |
| | Biotin | 0.015 |
| | B12 | 0.01 |
| | Calcium pantothenate | 0.03 |
| | p-Aminobenzoic acid | 0.06 |

Incubation is performed under the following conditions:
duration: 72 h;
temperature: 28° C.;
shaking: 110 rpm (Infors Multitron incubator).

The preculture is then transferred to a 30 L Sartorius type fermenter.

Culture for Biomass Production:

The medium is as follows:

TABLE 11

| | | |
|---|---|---|
| Macro | Glucose | 40 |
| elements (g/l) | KH₂PO₄ | 1.8 |
| | NaH₂PO₄ | 1.4 |
| | MgSO₄•7H₂O | 3.4 |
| | (NH₄)₂SO₄ | 0.2 |
| | clerol FBA 3107 (antifoam) | 0.3 |
| Microelements | CaCl₂•2H₂O | 40 |
| and Vitamins | FeSO₄•7H₂O | 12 |
| (mg/l) | MnSO₄•1H₂O | 40 |
| | CoSO₄•7H₂O | 0.1 |
| | CuSO₄•5H₂O | 0.5 |
| | ZnSO₄•7H₂O | 50 |
| | H₃BO₃ | 15 |
| | Na₂MoO₄•2H₂O | 2 |
| | Thiamine HCl | 6 |
| | Biotin | 0.1 |
| | B12 | 0.06 |
| | Calcium pantothenate | 0.2 |
| | p-Aminobenzoic acid | 0.2 |

The initial volume (Vi) of the fermenter is adjusted to 17 L after inoculation.

It is brought to a final volume of about 20-25 L.

The parameters for performing the fermentation are as follows:

TABLE 12

| | |
|---|---|
| Temperature | 28° C. |
| pH | 5.0-5.2 by 28% w/w NH₃ |
| pO₂ | 20% ± 5% (maintained by shaking) |
| Shaking | Minimum 300 rpm |
| Air flow rate | 15 L/min |

When the residual glucose concentration falls below 10 g/l, glucose in the form of a concentrated solution at approximately 800 g/l is introduced so as to maintain the glucose content between 0 and 20 g/l in the fermenter.

Results

The results are given as a function of time and of the C/P ratio which represents the amount of carbon consumed (originating from glucose) relative to the amount of phosphorus supplied (via phosphate).

TABLE 13

| Duration | C/P | Residual | Biomass | Cumulative | % N |
|---|---|---|---|---|---|
| (h) | (g/g) | PO₄ (g/L) | (g/l) | μ (h⁻¹) | 6.25 |
| 0.0 | | 2.25 | 3.0 | | — |
| 13.0 | 13.0 | 1.43 | 15.5 | 0.126 | — |
| 20.0 | 25.0 | 0.65 | 28.5 | 0.113 | 35.9 |
| 28.0 | 70.0 | 0.00 | 54.3 | 0.103 | 38.4 |
| 36.0 | 120.0 | 0.00 | 80.0 | 0.091 | 56.5 |
| 40.0 | 145.0 | 0.00 | 89.5 | 0.085 | 68.5 |

These results show that the protein content increases markedly at the end of fermentation from the moment when all the phosphate supplied is consumed and the C/P exceeds a value of 60.

As regards the energy, the cooling capacity of the exchanger of this fermenter, fed with water at 25° C., is at 1.7 kW/m² of exchange.

### Example 6: Production of Chlorella protothecoides at 31° C. in Fermentation of Fed-Batch Type Using a Phosphate-Limited Synthetic Medium

This test is performed under the same conditions as in the preceding example, except for the temperature, which is raised to 31° C. (instead of 28° C.).

Results

The results are given as a function of time and of the C/P ratio which represents the amount of carbon consumed (originating from glucose) relative to the amount of phosphorus supplied (via phosphate).

The cumulative μ corresponds to the growth rate of the biomass from inoculation.

The protein content is estimated by measuring the nitrogen content N×6.25.

TABLE 14

| Duration | C/P | Residual | Biomass | Cumulative | % N |
|---|---|---|---|---|---|
| (h) | (g/g) | PO₄ (g/L) | (g/L) | μ (h⁻¹) | 6.25 |
| 0.0 | | 2.25 | 3 | | |
| 13.0 | 11.2 | 1.54 | 13.5 | 0.102 | 42.9 |
| 20.0 | 23.7 | 0.83 | 26.7 | 0.098 | 36.6 |
| 28.0 | 60.1 | 0.00 | 51.7 | 0.101 | 35.1 |
| 36.0 | 99.3 | 0.00 | 70.2 | 0.09 | 51.2 |
| 40.0 | 121.6 | 0.00 | 79.8 | 0.085 | 58.9 |
| 44.0 | 143.9 | 0.00 | 88.8 | 0.079 | 71.9 |

These results first confirm those obtained at 28° C. Furthermore, increasing the temperature to 31° C. makes it possible to further reduce the growth rate and to increase the protein content (by about 5%).

Moreover, increasing the temperature to 31° C. instead of 28° C. makes it possible to very markedly improve the efficiency of the exchanger of the fermenter since this increases the temperature difference between the water feeding the exchanger and the fermentation must: the cooling capacity of the exchanger is raised to 3.5 kW/m² instead of 1.7 kW/m².

### Example 7: Amino Acid Composition of the Biomass of Chlorella protothecoides Produced Under Phosphate-Deficient Conditions

The total amino acid composition of the microalgal biomasses produced according to the method detailed in standard ISO 13903: 2005 is determined.

The following biomasses are analyzed:

Batch (A): biomass of Chlorella protothecoides produced according to the conditions of Example 6, and having a protein content of between 60% and 70% (expressed as N6×25).

Batch (B): biomass of Chlorella protothecoides produced according to the conditions of Example 5, and having a protein content of between 45% and 60% (expressed as N6×25).

Batch (C): biomass of Chlorella protothecoides prepared according to test 1 of Example 4, and having a protein content of between 45% and 50% (expressed as N6×25).

Batch (D): biomass of Chlorella sorokiniana produced according to the conditions of Example 3, and having a protein content of between 50% and 60% (expressed as N6×25).

Table 15 below has the total amino acid composition of the biomass, expressed in relative percentages.

TABLE 15

| | Batch A | Batch B | Batch C | Batch D |
|---|---|---|---|---|
| | relative % | relative % | relative % | relative % |
| Aspartic acid | 3.7 | 6.4 | 7.9 | 9.0 |
| Threonine | 1.9 | 4.0 | 4.8 | 4.7 |
| Serine | 1.9 | 3.5 | 4.5 | 4.0 |
| Glutamic acid | 36.3 | 22.3 | 15.3 | 11.5 |
| Glycine | 2.2 | 4.0 | 5.2 | 5.9 |
| Alanine | 4.1 | 6.4 | 8.0 | 8.6 |
| Valine | 2.4 | 4.7 | 6.0 | 5.9 |
| Isoleucine | 1.4 | 2.8 | 3.6 | 3.8 |
| Leucine | 3.4 | 6.6 | 8.2 | 8.7 |
| Tyrosine | 1.7 | 2.6 | 3.3 | 3.9 |
| Phenylalanine | 1.8 | 3.3 | 4.0 | 4.7 |
| Lysine | 2.5 | 4.4 | 5.3 | 8.8 |
| Histidine | 0.9 | 1.7 | 2.1 | 2.3 |
| Arginine | 31.4 | 20.1 | 12.8 | 6.8 |
| Proline | 1.8 | 3.4 | 4.7 | 5.2 |
| Cystine | 0.8 | 0.9 | 1.0 | 1.6 |
| Methionine | 0.9 | 1.5 | 1.7 | 2.2 |
| Tryptophan | 0.9 | 1.3 | 1.5 | 2.3 |
| TOTAL | 100.0 | 100.0 | 100.0 | 100.0 |

The results obtained clearly show that, on the total amino acid composition of the Chlorella protothecoides biomasses, more than 40% (in relative terms) of the amino acids are glutamic acid and arginine if Chlorella protothecoides is cultured under conditions that enrich its protein content (not more than 30% under standard culture conditions—cf. Batch C).

Moreover, this result is obtained only for Chlorella protothecoides, which tends to show the particular metabolic features of this microalga, with regard to Chlorella sorokiniana. Specifically, although having a high protein content, C. sorokiniana—Batch D—does not produce more than 20% of glutamic acid and arginine.

1.-15. (canceled)

16. A process for the protein enrichment of a microalga cultured heterotrophically in a fermentation medium, the microalga being of the genus Chlorella, wherein the heterotrophic culturing comprises a step of limiting a non-nitrogen nutrient in the fermentation medium, and wherein the protein content of the microalga is more than 50% by weight.

17. The process according to claim 16, wherein the non-nitrogen nutrient is a nutrient selected from the group consisting of glucose and phosphate.

18. The process according to claim 16, wherein the microalga pf the genus Chlorella is chosen from Chlorella sorokiniana or Chlorella protothecoides.

19. The process according to claim 16, wherein the heterotrophic culturing of the microalgae of the species comprises:
a) a first step of growth of the microalgae, in which the glucose supply is not limiting; and
b) a second step wherein the glucose supply is limited, wherein the limited glucose supply reduces the additional accumulation of storage substances.

20. The process according to claim 16, wherein the heterotrophic culturing of the microalgae of the species Chlorella protothecoides comprises a step of culturing with limiting a phosphate nutrient, wherein the growth rate is reduced and an increase in the protein content is obtained.

21. The process according to claim 20, wherein the heterotrophic culturing of the microalgae of the species Chlorella protothecoides comprises a step of culturing with limiting a phosphate nutrient, wherein the growth rate is reduced and an increase in the protein content is obtained, and further wherein the combined amount of glutamic acid and arginine together in the protein is more than 40%.

22. The process according to claim 16, wherein the fermentation temperature is modified to reduce the growth rate of the microalga.

23. The process according to claim 22, wherein the fermentation temperature is increased by about 1-5° C. relative to the optimum fermentation temperature.

24. The process according to claim 23, wherein the fermentation temperature is increased by about 3° C. relative to the optimum fermentation temperature.

25. The process according to claim 23, wherein the fermentation temperature increase leads to increasing the cooling capacity of the heat exchanger of the fermenter.

26. The process according to claim 16, wherein the fermentation temperature is about 31° C.

27. A process for the protein enrichment of a microalga cultured heterotrophically in a fermentation medium, wherein the heterotrophic culturing comprises a step directed towards slowing down the growth of said microalga by increasing the fermentation temperature relative to the optimum fermentation temperature, and limiting a non-nitrogen nutrient in the fermentation medium, and wherein the protein content of the microalga is more than 50% by weight.

28. The process according to claim 27, wherein the increased fermentation temperature is from 28° C. to 31° C.

29. The process according to claim 27, wherein the microalga is of the species Chlorella protothecoides.

30. A process for enriching the glutamic acid and/or arginine content of a heterotrophically cultivated microalga, the process comprising heterotrophic cultivation of the microalga in a fermentation medium, the fermentation medium comprising a limiting amount of a phosphate nutrient, and wherein the combined glutamic acid and arginine content of the protein content of the microalga is more than 40%.

31. The process a according to claim 30, wherein the microalga are of the species Chlorella protothecoides.

32. A microalgal biomass obtained via the process of claim 30.
